(19) United States
(12) Patent Application Publication
(10) Pub. No.: US 2026/0360489 A1
(43) Pub. Date: Nov. 12, 2026

(54) Generating Electronic Tumor Marker Analogs of Carbohydrate Antigen 19-9 Using Machine Learning

(71) Applicant: The Medical College of Wisconsin, Inc., Milwaukee, WI (US)

(72) Inventors: Anai N. Kothari, Brookfield, WI (US); Sam Z. Thalji, Milwaukee, WI (US)

(21) Appl. No.: 19/111,968

(22) PCT Filed: Sep. 18, 2023
(86) PCT No.: PCT/US2023/074508

§ 371 Date: Mar. 14, 2025

Related U.S. Application Data
(60) Provisional application No. 63/407,279, filed on Sep. 16, 2022.

(51) Int. Cl. G16H 15/00; G06N 20/00; G16H 10/40; G16H 10/60

(52) U.S. Cl. CPC G16H 15/00; G06N 20/00; G16H 10/40; G16H 10/60

(57) ABSTRACT

Electronic tumor marker values are generated from patient health data using a suitably trained machine learning model. In general, the electronic tumor marker data include estimated values of an electronic tumor marker that is analogous or otherwise complementary to carbohydrate antigen 19-9 (“CA19-9”) biomarkers measured from a patient or biological specimen.

### BACKGROUND

Tumor markers can detect cancer, guide treatment, and identify recurrence. Existing tumor markers are biologic and must be obtained through direct sampling of a patient's blood or other tissue. Pancreatic ductal adenocarcinoma (“PDAC”) is the third leading cause of cancer-related death in the United States and estimated to become the second leading cause by 2030. It generally is detected at an advanced stage and measuring response to anticancer therapy is difficult because of the unique biologic properties of the tumor.

Presently, the most commonly used tumor marker for PDAC is carbohydrate antigen 19-9 (“CA19-9”) which is a tetrasaccharide cell-surface molecule expressed incidentally by some GI cancers. Measuring an individual patient's CA19-9 level requires a blood draw and assessment of the amount of CA19-9 in the blood. This is done for nearly all patients at risk for or with a diagnosis of PDAC, and modern therapy of pancreas cancer relies heavily on CA19-9 assessment. However, up to 30% of patients with PDAC do not secrete CA19-9 at elevated or informative levels, including 10% of all patients who do not secrete CA19-9 at any detectable level. For patients without elevated CA19-9, the lack of biochemical information represents a “missing value” in the ability to assess treatment response.

### SUMMARY OF THE DISCLOSURE

The present disclosure addresses the aforementioned drawbacks by providing a method for generating an electronic tumor marker from patient health data. The method includes accessing with a computer system, patient health data acquired from a patient. A machine learning model that has been trained on training data in order to estimate an electronic tumor marker value from patient health data is also accessed with the computer system. The patient health data are input to the machine learning model using the computer system, generating output data as electronic tumor marker data comprising electronic tumor marker values that are predictive of, or otherwise analogous to, carbohydrate antigen 19-9 (CA19-9) serum values in the patient. The electronic tumor marker data are then presented to a user.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for generating electronic tumor marker (e19-9) values from patient health data using a suitably trained machine learning model.

FIG. 2 is a flowchart setting forth the steps of an example method for training a machine learning model to generate electronic tumor marker (e19-9) values from patient health data.

FIGS. 3A and 3B illustrate ROC curves for predicting the completion of all neoadjuvant treatment and surgery from an example study. Receiver-operating characteristic (ROC) curves for the outcome of completion of all neoadjuvant treatment and surgery: (a) represents the proportional change in e19-9 from pre-treatment (diagnosis) to post-treatment (preoperative); (b) represents the value of the post-treatment e19-9.

FIG. 4 illustrates a table of logistic regression models for completion of all neoadjuvant treatment and surgery.

FIGS. 5A-5C illustrate overall survival from diagnosis among all patients in an example study. The median overall survival among all patients (n=121) was 45 months. (a) The median overall survival among patients that achieved at least a 50% decline in e19-9 from pre-treatment (diagnosis) to post-treatment (preoperative) (n=62) was 53 months compared to 32 months among patients without a 50% decline (n=59) (p=0.007). (b) The median overall survival among patients with a post-treatment e19-9 below 100 (n=88) was 60 months compared to 16 months among patients with post-treatment e19-9≥100 (n=33) (p<0.001). FIG. 5C shows the median overall survival among patients that completed neoadjuvant treatment and surgery (n=93) was 66 months: 68 months among patients with a post-treatment (preoperative) e19-9 below 100 (n=81) and 38 months among patients with post-treatment e19-9≥100 (n=12) (p=0.04).

FIG. 6 illustrates a table of Cox proportional hazards models for survival.

FIGS. 7A and 7B illustrate scatterplots of predicted e19-9 values and real CA19-9 values. The CA19-9 values among patients in the internal and external datasets compared to predicted e19-9 values. Values in the illustrated plots are log₁₀ transformed. FIG. 7A shows results for the trained e19-9 model on an internally withheld test set (75/25 split), which had performance metrics that were RMSE 0.739 and R² 0.365. FIG. 7B shows results for the trained e19-9 model on the external dataset, which has performance metrics that were RMSE 0.667 and R² 0.496.

FIG. 8 is a block diagram of an example system for generating electronic tumor marker data from patient health data.

FIG. 9 is a block diagram of example components that can implement the system of FIG. 8.

### DETAILED DESCRIPTION

Described here are systems and methods for generating or otherwise estimating electronic tumor marker values from patient health data using a suitably trained machine learning model. In general, the electronic tumor marker values include an electronic tumor marker that is analogous or otherwise complementary to carbohydrate antigen 19-9 (“CA19-9”) biomarker values measured from a patient or biological specimen. In this way, the electronic tumor marker value may be referred to as e19-9 electronic tumor marker value, which may include estimated e19-9 values that are analogous, complementary, or otherwise indicative of CA19-9 values.

The widespread use of electronic health records has generated an enormous amount of patient data and facilitated the linkage of genotypic and research data; this pooling of siloed information allows machine learning models to expose previously unknown associations that predict clinical outcomes. Supervised machine learning algorithms have been shown to accurately stratify risk and prognosis, aid in early detection, and predict treatment response for a variety of diseases. Patients with PDAC that lack informative CA19-9 levels still possess a library of clinical datapoints that may shed light on their underlying tumor biology. A machine learning model trained to predict the value of a well-established tumor biomarker could utilize this wealth of information. A predicted biomarker using available data would in itself be prognostic for patients with non-informative CA19-9 and replace the missing values on which we are increasingly basing treatment decisions for PDAC.

Advantageously, the e19-9 values generated using the systems and methods described in the present disclosure can measure clinically relevant medical conditions and data among patients who do not produce biochemical tumor markers to provide insight into the disease burden and response to treatment. As a result, clinicians can analyze or review the electronic tumor marker data to inform clinical decisions including treatment approach. Additionally or alternatively, the electronic tumor markers may change or rise before elevated levels of CA19-9, or other biochemical tumor markers, can be detected in some patient cohorts. As a result, the systems and methods described in the present disclosure can generate electronic tumor marker values that a clinician can analyze or otherwise review to assist in making diagnoses of disease, such as pancreatic cancer. For instance, in an example study it was observed that in PDAC patients who do not produce CA19-9, the electronic tumor marker e19-9 was able to measure tumor response with near identical performance to the serum biologic version. Electronic tumor markers represent a previously non-described approach to screen, diagnose, and identify cancer recurrence. This is accomplished without needing a special test or assay, and can be calculated with patient health data that is commonly collected during routine care.

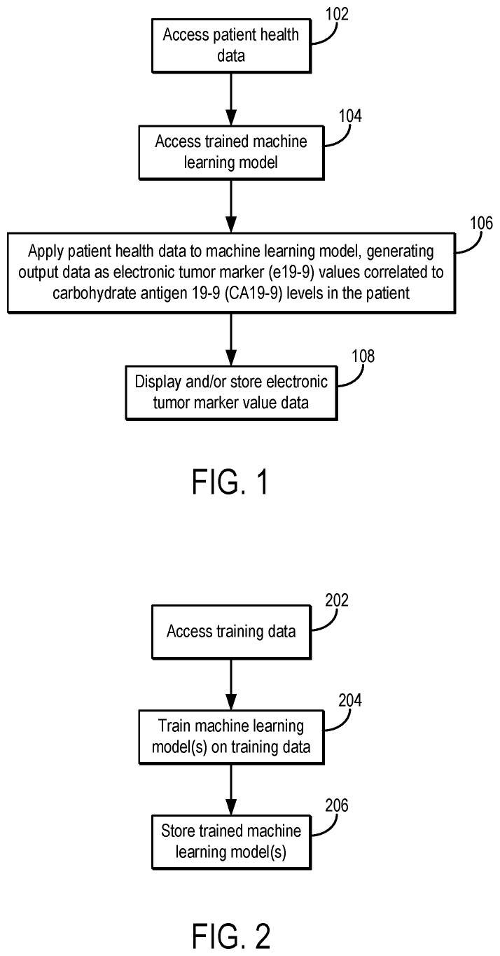

Referring now to FIG. 1, a flowchart is illustrated as setting forth the steps of an example method for generating electronic tumor marker values using a trained machine learning model. As will be described, the machine learning model, which in some instances may be a neural network, takes patient health data as input data and generates electronic tumor marker values as an output. As an example, the electronic tumor marker value can be an electronic tumor marker that is complementary to a CA19-9 biomarker. This electronic tumor marker may be referred to as an e19-9 electronic tumor marker. In some implementations, the machine learning models described in the present disclosure implement regression in order to build a framework where the predicted electronic tumor marker values output by the model may be dynamically followed during treatment to allow for real-time clinical decision-making.

The method includes accessing patient health data with a computer system, as indicated at step 102. Accessing the patient health data may include retrieving such data from a memory or other suitable data storage device or medium.

In general, the patient health data can include data stored in, retrieved from, extracted from, or otherwise derived from the patient's electronic medical record (“EMR”) and/or electronic health record (“EHR”). The patient health data can include unstructured text, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

Features derived from structured, curated, and/or EMR or EHR data may include clinical features such as diagnosis, symptoms, therapies, outcomes, patient demographics such as patient name; date of birth; gender; ethnicity; diagnosis dates for cancer, illness, disease, or other physical or mental conditions; personal medical history; family medical history; clinical diagnoses, such as date of initial diagnosis, date of metastatic diagnosis, cancer staging, tumor characterization, and tissue of origin. Additionally, the patient health data may also include features such as treatments and outcomes, such as line of therapy, therapy groups, clinical trials, medications prescribed or taken, surgeries, radiotherapy, imaging, adverse effects, and associated outcomes.

Patient clinical data can include a set of clinical features associated with information derived from clinical records of a patient, which can include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EMR and/or EHR.

In some instances, patient health data can include medical imaging data, which may include images of the patient obtained with one or more different medical imaging modalities, including magnetic resonance imaging (“MRI”), computed tomography (“CT”), x-ray imaging, positron emission tomography (“PET”), ultrasound, and so on. The medical imaging data may also include parameters or features computed or derived from such images. Medical imaging data may also include digital pathology images, such as H&E slides, IHC slides, and the like. The medical imaging data may also include data and/or information from pathology and radiology reports, which may be ordered by a physician during the course of diagnosis and treatment of various illnesses and diseases.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

In some embodiments, the patient health data can include a collection of data and/or features including all of the data types disclosed above. Alternatively, the patient health data may include a selection of fewer data and/or features.

A trained machine learning model is then accessed with the computer system, as indicated at step 104. Alternatively, the machine learning model may be an artificial intelligence model, engine, or analytics framework. Accessing the machine learning model may include accessing model parameters (e.g., weights, biases, or both) that have been optimized or otherwise estimated by training the machine learning model on training data. In some instances, retrieving the machine learning model can also include retrieving, constructing, or otherwise accessing the particular model architecture to be implemented. For instance, when the machine learning model includes an artificial neural network, data pertaining to the layers in the neural network architecture (e.g., number of layers, type of layers, ordering of layers, connections between layers, hyperparameters for layers) may be retrieved, selected, constructed, or otherwise accessed. As another example, when the machine learning model includes a random forest, data pertaining to the model architecture (e.g., number of decision trees in the forest, number of features considered by each tree when splitting a node, minimum/maximum sample leaf size, etc.) may be retrieved, selected, constructed, or otherwise accessed.

In general, the machine learning model is trained, or has been trained, on training data in order to estimate an electronic tumor marker, electronic tumor marker, or the like, such as an e19-9 electronic tumor marker. In some implementations, the patient health data are processed using a dimensionality reduction technique in order to reduce the feature set (e.g., from thousands to tens of features) prior to inputting the patient health data to the machine learning model. Alternatively, the dimensionality reduction may be a part of the machine learning model. Performing dimensionality reduction without losing information may be implemented using unsupervised or supervised learning techniques. Unsupervised methods may include singular value decomposition (“SVD”), principal component analysis (“PCA”), k-means clustering, and so on. Performing dimensionality reduction in a supervised manner may include linear discriminant analysis, neighborhood component analysis, tree based supervised embedding, and so on.

Machine learning models or algorithms can be supervised and/or unsupervised. As a non-limiting example, supervised models can include models where the features and/or classifications in the dataset are annotated, and can include those models using linear regression, logistic regression, decision trees, classification, regression trees, naive Bayes, nearest neighbor clustering, and the like. Unsupervised models can include models where no features and/or classifications in the dataset are annotated, and can include those models using a priori, means clustering, PCA, random forest, adaptive boosting, and the like. Machine learning models may also be semi-supervised (e.g., where an incomplete number of features and/or classifications in the dataset are annotated), and may include generative approaches (e.g., a mixture of Gaussian distributions, mixture of multinomial distributions, hidden Markov models), low density separation, graph-based approaches (e.g., mincut), heuristic approaches, or support vector machines (“SVMs”).

In some implementations, the machine learning model may be an artificial neural network. An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

The patient health data are then input to the one or more trained machine learning models, generating an output as electronic tumor marker values, as indicated at step 106. For example, the output may include one or more electronic tumor marker values, such as e19-9 electronic tumor marker values. As another example, the model output may indicate the probability for a particular classification (i.e., the probability that the patient health data and/or e19-9 values estimated therefrom include patterns, features, or characteristics indicative of detecting, differentiating, and/or determining the severity of one or more medical conditions). Additionally or alternatively, the model output may classify the patient health data as indicating a particular medical condition, which in some instances the classification may be based on the estimated e19-9 values. In these instances, the electronic tumor marker value can differentiate between different medical conditions. In still other embodiments, the model output may indicate a severity of a medical condition based on the estimated e19-9 electronic tumor marker values. For example, the model output may include a severity score that quantifies a severity of a medical condition, or a severity score can be estimated or derived from estimated electronic tumor marker values.

The electronic tumor marker value generated by inputting the patient health data to the trained machine learning model(s) can then be displayed to a user, stored for later use or further processing, or both, as indicated at step 108. For example, the electronic tumor marker value may be displayed or otherwise presented to a user via a computer system. Displaying the electronic tumor marker value may include presenting the electronic tumor marker value as a numerical display, a text display, and the like. In some examples, outputting the electronic tumor marker value may include generating a report that includes the electronic tumor marker value and displaying the report to a user. Additional data may also be presented in the report, such as some or all of the patient health data, or other data or information associated with the patient. In still other instances, outputting the electronic tumor marker value may include storing the electronic tumor marker value in the patient's EMR and/or EHR.

As noted above, the electronic tumor marker value can be predictive of a medical condition in the patient. For example, the electronic tumor marker value may be predictive of the patient having pancreatic cancer. In these instances, outputting the electronic tumor marker value may also include outputting a predictive value or indication that the patient is at risk for, or has, a particular medical condition, such as pancreatic cancer. For example, a report may be generated, where the report indicates a predictive value or indication that the patient is at risk for, or has, a particular medical condition, such as pancreatic cancer.

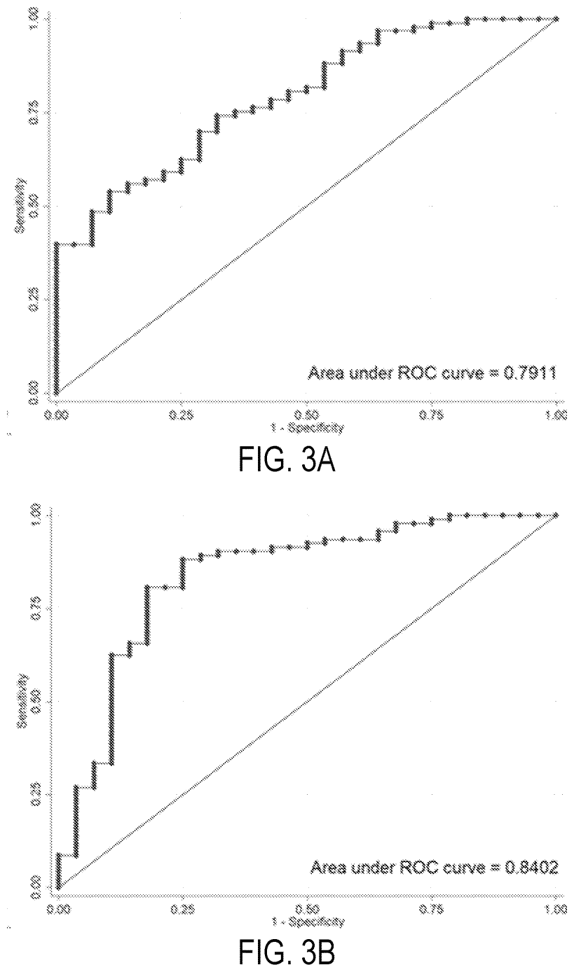

Referring now to FIG. 2, a flowchart is illustrated as setting forth the steps of an example method for training one or more machine learning models on training data, such that the one or more machine learning models are trained to receive patient health data as input data in order to generate one or more electronic tumor marker values as output data, where the electronic tumor marker values are indicative of an electronic tumor marker, such as an e19-9 electronic tumor marker. In general, the machine learning model(s) can implement any number of different model architectures, such as those described above.

The method includes accessing training data with a computer system, as indicated at step 202. Accessing the training data may include retrieving such data from a memory or other suitable data storage device or medium. For instance, the training data may be retrieved from one or more data stores or content servers, which in some instances may include an EMR server, an EHR server, a radiology information server (“RIS”), and so on.

In general, the training data can include patient health data obtained from one or more groups of patients, controls, or other populations. In some embodiments, the training data may include patient health data sets or features derived from patient health data sets that have been labeled (e.g., labeled as containing patterns, features, or characteristics, and the like).

The method can include assembling training data from patient health data using a computer system. This step may include assembling the patient health data into an appropriate data structure on which the neural network or other machine learning algorithm can be trained. Assembling the training data may include assembling patient health data and other relevant data. For instance, assembling the training data may include generating labeled data and including the labeled data in the training data. Labeled data may include patient health data or other relevant data that have been labeled as belonging to, or otherwise being associated with, one or more different classifications or categories.

One or more machine learning models are trained on the training data, as indicated at step 204. In general, the machine learning model can be trained by optimizing model parameters (e.g., weights, biases, or both) based on minimizing a loss function. As one non-limiting example, the loss function may be a mean squared error loss function.

As an example, training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as electronic tumor marker value. The artificial neural network then compares the generated output with the actual output of the training example in order to evaluate the quality of the electronic tumor marker value. For instance, the electronic tumor marker value can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

Additionally or alternatively, training the machine learning model may include providing optimized datasets as a matrix of feature vectors for each patient, labeling these features as they occur in patient records, and training the machine learning model to predict an objective/target pairing.

The one or more trained machine learning models are then stored for later use, as indicated at step 206. Storing the machine learning model(s) may include storing model parameters (e.g., weights, biases, or both), which have been computed or otherwise estimated by training the machine learning model(s) on the training data. Storing the trained machine learning model(s) may also include storing the particular model architecture to be implemented. For instance, when the machine learning model is a neural network, data pertaining to the layers in the neural network architecture (e.g., number of layers, type of layers, ordering of layers, connections between layers, hyperparameters for layers) may be stored.

Described now is an example application of the analytical framework for generating and monitoring an e19-9 electronic tumor marker from patient health data of patients that do not otherwise produce CA19-9 biomarkers (e.g., in a biological serum).

The e19-9 values were estimated using a trained model in a cohort of patients with known clinical metrics to assess the reliability of e19-9 in determining commonly studied outcomes for PDAC. Inclusion criteria were 1) diagnosis of anatomically resectable or borderline resectable PDAC; 2) received neoadjuvant anticancer therapy (chemotherapy and/or radiotherapy) prior to intended surgical resection; 3) did not produce an elevated CA19-9 level (>35 U/mL) at any time point during their diagnosis or treatment of localized PDAC; 4) lab data available for model input at the time of diagnosis and at the time of completion of neoadjuvant therapy (prior to intended surgery). Patients with missing lab data (usually due to treatment at another institution prior to referral) were excluded. These patients, by definition of not producing CA19-9 at elevated levels, were confirmed to have not been used in the training of the e19-9 model.

A total of 121 patients were identified: 63 patients with anatomically resectable disease and 58 patients with borderline resectable disease. All 121 patients received anticancer therapy prior to intended surgery: 8 received chemotherapy alone, 37 received radiotherapy alone, and 76 received both. Among all 121 patients, the median pretreatment CA19-9 was 11.3 U/mL (IQR 18.1) and the median post-treatment (preoperative) CA19-9 was 8.7 U/mL (IQR 13.8). The e19-9 model was applied to these patients for all available time points in which labs were available. In this cohort of patients with low uninformative CA19-9 levels, e19-9 was predicted over a wide range that did not correlate with CA19-9. Among all 121 patients, the median pretreatment e19-9 was 124.7 (IQR 181) and the median post-treatment (preoperative) e19-9 was 60.6 (IQR 72). The median proportional change in e19-9 over treatment among all 121 patients was-51.5% (IQR 67.3). Among all 121 patients, 91 (75%) had a decline in e19-9 of any magnitude over the course of treatment.

TABLE 1
Demographics and Pre-treatment Tumor Factors by Completion
of All Neoadjuvant Treatment and Surgery

| | | | Completed NeoTx + | | Did Not Complete | | |
|---|---|---|---|---|---|---|---|
| | Total (n = 121) | | Surg (n = 93) | | (n = 28) | | p value |
| Age in years, median (IQR) | 65 | (14) | 65 | (14) | 62.5 | (13) | 0.05 |
| Gender (female), n (%) | 59 | (49) | 11 | (39) | 48 | (52) | 0.25 |
| Race (n) | | | | | | | 0.14 |
| White | 108 | (89) | 85 | (91) | 23 | (82) | |
| Black | 11 | (9) | 6 | (6) | 5 | (18) | |
| Other | 2 | (2) | 2 | (2) | 0 | | |
| BMI (kg/m\hat{ }2), median (IQR) | 27.0 | (6.0) | 26.8 | (5.3) | 27.8 | (7.7) | 0.63 |
| Charlson comorbidity index, median | 5 | (2) | 5 | (2) | 4.5 | (2) | 0.30 |
| (IQR) | | | | | | | |
| Clinical stage, n (%) | | | | | | | 0.02 |
| Respectable | 63 | (52) | 52 | (58) | 9 | (32) | |
| Borderline Respectable | 58 | (48) | 39 | (42) | 19 | (68) | |
| Tumor Size on Dx Imaging (cm), | 2.6 | (1.7) | 2.6 | (1.6) | 2.7 | (2.6) | 0.54 |
| median (IQR) | | | | | | | |
| Neoadjuvant Therapy, n (%) | | | | | | | 0.77 |
| Both | 76 | (63) | 57 | (61) | 19 | (68) | |
| Chemotherapy alone | 8 | (7) | 6 | (6) | 2 | (7) | |
| Radiotherapy alone | 37 | (31) | 30 | (32) | 7 | (25) | |
| CA19-9 at Dx (U/ml), median | 11.3 | (18.1) | 12.3 | (19.0) | 2.6 | (13.4) | 0.03 |
| (IQR) | | | | | | | |
| e19-9 at Dx, median (IQR) | 124.7 | (181) | 121.0 | (197) | 134.1 | (151) | 0.96 |

Among the 121 patients, 93 (77%) patients completed all neoadjuvant treatment including surgery while 28 (23%) patients did not. The reasons for deferring surgery among these 28 patients included the development of metastatic disease (discovered at the time of restaging imaging or diagnostic laparoscopy) in 17 patients, local disease progression in 2 patients, inadequate performance status in 8 patients, and patient choice in 1 patient. Surgical resection was accomplished among 54 (86%) of the 63 anatomically resectable patients and 39 (67%) of the 58 borderline resectable patients (p=0.02). There were no other significant differences in patient or disease related factors at diagnosis between patients who did and did not complete neoadjuvant treatment and surgery (Table 1). The pre-treatment e19-9 was 121.0 (IQR 197) among the 93 patients that completed treatment and surgery and 134.1 (IQR 151) among the 28 patients that did not (p=0.96). The median proportional change in e19-9 between pre-treatment and post-treatment values was −53.7% (IQR 59.1) among the 93 patients who completed treatment and surgery compared to −1.8% (IQR 131.9) among the 28 patients who did not (p<0.001). Among the 93 patients who completed treatment and surgery, 76 (82%) had a decline in e19-9 of any magnitude compared to 15 (54%) of the 28 patients who did not complete surgery (p=0.005). The median post-treatment e19-9 was 47.9 (IQR 37) among the 93 patients who competed treatment and surgery compared to 147.2 (IQR 192) among the 28 patients who did not (p<0.001). Analyses of absolute values and trends of CA19-9 over the treatment period in this cohort were not informative in predicting the completion of all neoadjuvant treatment and surgery.

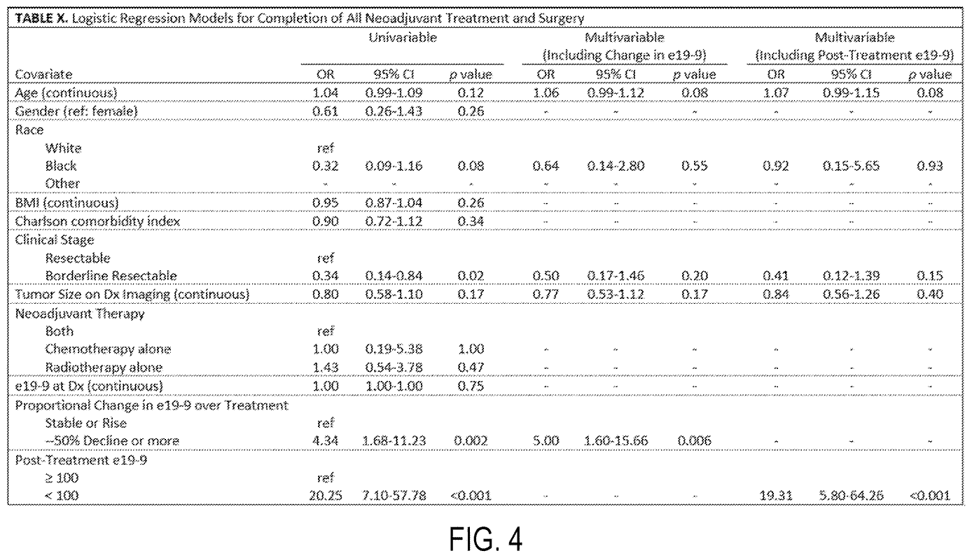

An ROC curve for the proportional change in e19-9 over treatment for predicting completion of neoadjuvant treatment and surgery is shown in FIG. 3A (AUC 0.79). The optimal cutpoint determined using the Youden index was a −52.6% decline in e19-9 from pre-treatment to post-treatment (sensitivity 53.8%, specificity 89.3%, PPV 94.3%, NPV 36.8%). An ROC curve for the post-treatment e19-9 value for predicting completion of neoadjuvant treatment and surgery is shown in FIG. 3B (AUC 0.84). The optimal cutpoint determined using the Youden index was a post-treatment e19-9 value of 107.9 (sensitivity 88.2%, specificity 71.4%, PPV 91.1%, NPV 64.5%). Analysis of further outcomes incorporates rounded endpoints based on these ROC curves.

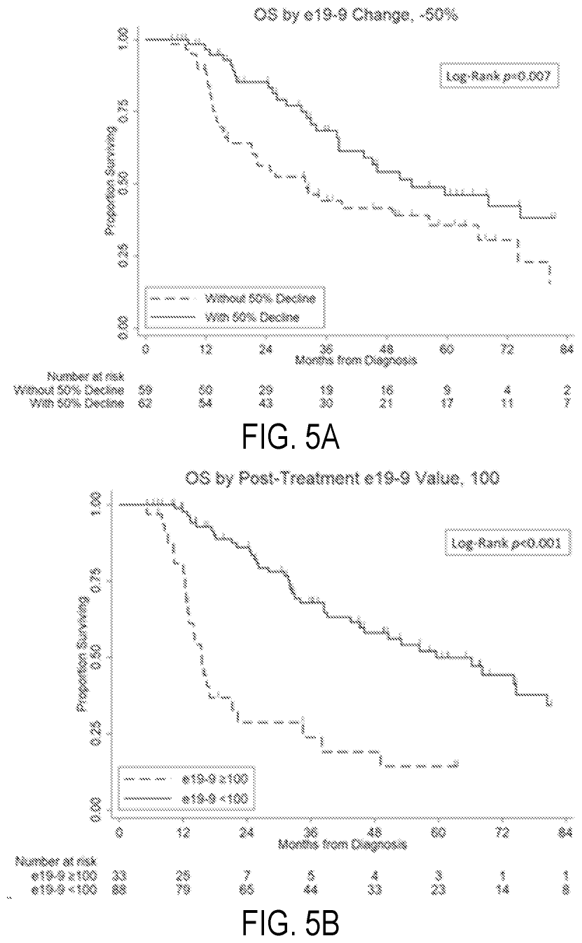

Univariable and multivariable models for factors associated with completion of all neoadjuvant treatment and surgery are displayed in the table shown in FIG. 4. On a multivariable model controlling for patient, disease, and treatment-related factors, a decline in e19-9 by at least −50% over treatment was independently associated with 5-fold increased odds of completing treatment and surgery (p=0.006). On a separate multivariable model controlling for the same factors, a post-treatment e19-9 below 100 was independently associated with 19.3-fold increased odds of completing treatment and surgery (p<0.001).

Among the 121 patients, 17 (14%) developed metastatic disease progression which was discovered at the time of restaging imaging or diagnostic laparoscopy. The site of metastasis was liver in 10 patients, lung in 5 patients, and peritoneum in 2 patients. These patients did not undergo surgical resection. The median pre-treatment e19-9 was 143.5 (IQR 114) among the 17 patients with metastatic progression compared to 121.8 (IQR 208) among the 104 patients without metastatic progression (p=0.77). The median change in e19-9 from pre-treatment to post-treatment was an increase in +26.4% (IQR 152) among the 17 patients with metastatic progression compared to −52.7% among the 104 without metastatic progression (p=0.004). The median post-treatment e19-9 was 128.5 (IQR 172) among the 17 patients with metastatic progression compared to 53.3 (IQR 51) among the 104 without metastatic progression (p=0.001). A decline in e19-9 of any magnitude was observed in 8 (47%) of the 17 patients with metastasis compared to 83 (80%) of patients without metastasis (p=0.01). A decline in e19-9 by at least-50% was observed in 4 (24%) of the 17 patients with metastasis compared to 58 (56%) of patients without metastasis (p=0.01). The post-treatment e19-9 was below 100 in 5 (29%) of 17 patients with metastasis compared to 83 (80%) of 104 patients without metastasis (p<0.001). Analyses of absolute values and trends of CA19-9 over the treatment period in this cohort were not informative in predicting metastatic progression during neoadjuvant treatment.

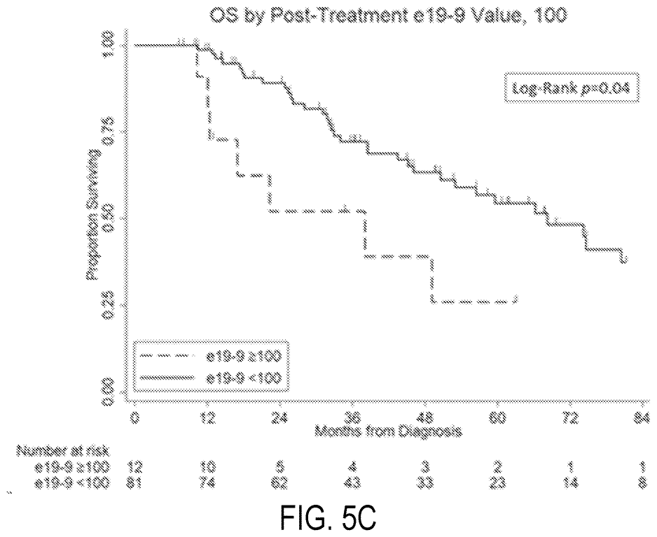

Among all 121 patients, the median OS from diagnosis was 45 months: 66 months among the 93 patients who completed neoadjuvant treatment and surgery compared to 15 months among the 28 patients who did not. The median OS for patients with pre-treatment e19-9 above and below the median of 124.7 were 45 and 39 months, respectively (p=0.78). The median OS among the 91 patients with a decline in e19-9 of any magnitude was 49 months compared to 22 months among the 30 patients stable or rising e19-9 (p=0.03). The median OS among the 62 patients who experienced at least a −50% decline in e19-9 over treatment was 53 months compared to 32 months among patients who did not meet a −50% decline (FIG. 5A, p=0.007). The median OS among the 88 patients with post-treatment e19-9 below 100 was 60 months compared to 16 months among the 33 patients with post-treatment e19-9 at or above 100 (FIG. 5B, p<0.001). When examining only the 93 patients that completed neoadjuvant treatment and surgery, the median OS among 81 patients with post-treatment e19-9 below 100 was 68 months compared to 38 months among the 12 patients with post-treatment e19-9 at or above 100 (FIG. 5C, p=0.04).

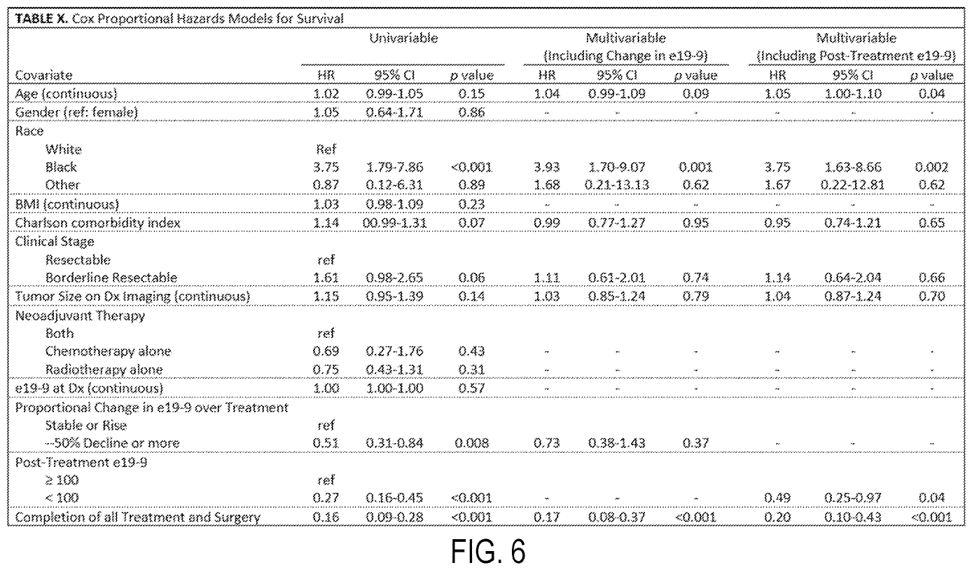

Univariable and multivariable cox proportional hazards models for survival are displayed in the table shown in FIG. 6. In separate multivariable models, greater proportional decline in e19-9 over treatment and lower post-treatment e19-9 were each independently associated with improved survival (not shown). The endpoint of a −50% decline in e19-9 over treatment did not reach significance (HR 0.73, p=0.37). The endpoint of post-treatment e19-9 below 100 was independently associated with improved survival (HR 0.49, p=0.04). Analyses of absolute values and trends of CA19-9 over the treatment period in this cohort were not associated with survival.

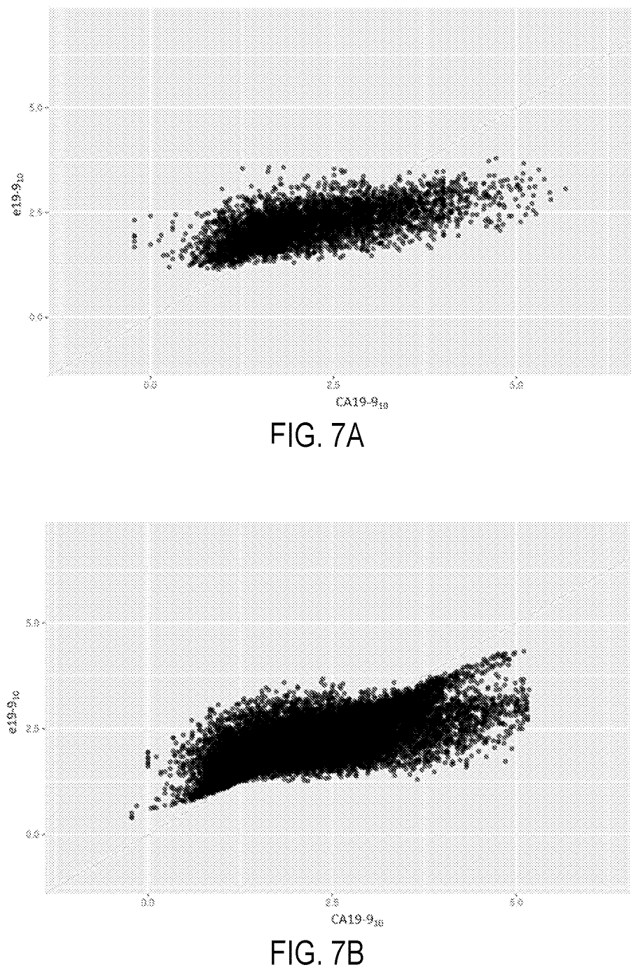

In another example, patient health data were obtained from patients with pancreatic cancer from 58 healthcare organizations. Patient datapoints were organized in the same manner as the training set in which all results were linked to a CA19-9 lab for the same patient on the same day. The external validation cohort included 4,384 unique patients with 16,487 total observations and was given to the model to generate an e19-9 value for each observation, as illustrated in FIGS. 7A and 7B. FIGS. 7A and 7B illustrate scatterplots of predicted e19-9 values and real CA19-9 values. The CA19-9 values among patients in the internal and external datasets compared to predicted e19-9 values. Values in the illustrated plots are log₁₀ transformed. FIG. 7A shows results for the trained e19-9 model on an internally withheld test set (75/25 split), which had performance metrics that were RMSE 0.739 and R² 0.365. FIG. 7B shows results for the trained e19-9 model on the external dataset, which has performance metrics that were RMSE 0.667 and R² 0.496.

The systems and methods described in the present disclosure provide for an electronic tumor marker (e19-9) that can feasibly be generated using a machine learning algorithm with readily available patient health data. The e19-9 electronic tumor marker correlates with an established serum tumor marker (CA19-9) among patients with PDAC that secrete it, as well as with important clinical outcomes among patients that do not secrete CA19-9 at informative levels. The results indicate that a machine learning model-derived electronic tumor marker for PDAC can provide valuable and relevant information to the nearly 30% of patients with PDAC who are missing a biochemical marker for assessment of prognosis and treatment response.

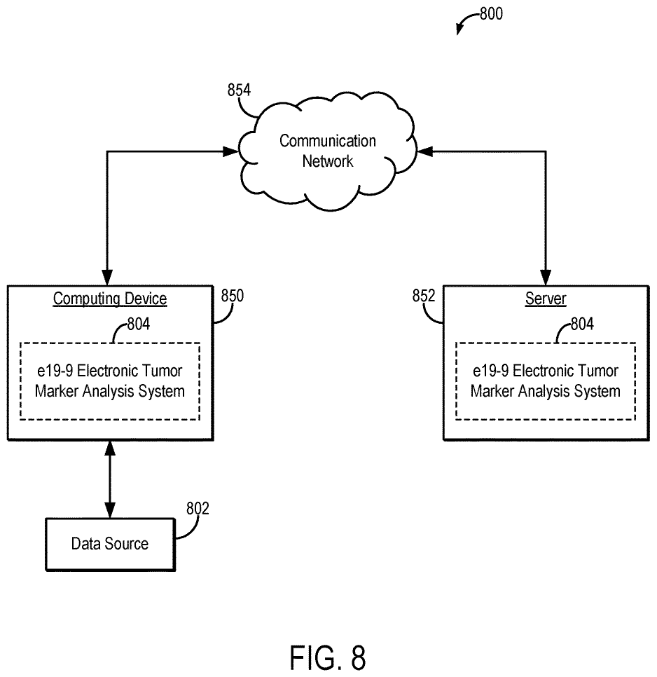

Referring now to FIG. 8, an example of a system 800 for generating electronic tumor marker data from patient health data in accordance with some embodiments of the systems and methods described in the present disclosure is shown. As shown in FIG. 8, a computing device 850 can receive one or more types of data (e.g., patient health data, such as those types of patient health data and features described above in more detail) from data source 802. In some embodiments, computing device 850 can execute at least a portion of an e19-9 electronic tumor marker analysis system 804 to generate and analyze e19-9 electronic tumor marker values from patient health data received from the data source 802.

Additionally or alternatively, in some embodiments, the computing device 850 can communicate information about data received from the data source 802 to a server 852 over a communication network 854, which can execute at least a portion of the e19-9 electronic tumor marker analysis system 804. In such embodiments, the server 852 can return information to the computing device 850 (and/or any other suitable computing device) indicative of an output of the e19-9 electronic tumor marker analysis system 804.

In some embodiments, computing device 850 and/or server 852 can be any suitable computing device or combination of devices, such as a desktop computer, a laptop computer, a smartphone, a tablet computer, a wearable computer, a server computer, a virtual machine being executed by a physical computing device, and so on. The computing device 850 and/or server 852 can also be a part of, or can communicate with, existing electronic health systems or modules.

In some embodiments, data source 802 can be any suitable source of data (e.g., patient health data), such as EMR and/or EHR data stores, another computing device (e.g., a server patient health data), and so on. In some embodiments, data source 802 can be local to computing device 850. For example, data source 802 can be incorporated with computing device 850 (e.g., computing device 850 can be configured as part of a device for measuring, recording, estimating, acquiring, or otherwise collecting or storing data). As another example, data source 802 can be connected to computing device 850 by a cable, a direct wireless link, and so on. Additionally or alternatively, in some embodiments, data source 802 can be located locally and/or remotely from computing device 850, and can communicate data to computing device 850 (and/or server 852) via a communication network (e.g., communication network 854).

In some embodiments, communication network 854 can be any suitable communication network or combination of communication networks. For example, communication network 854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 854 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, and so on.

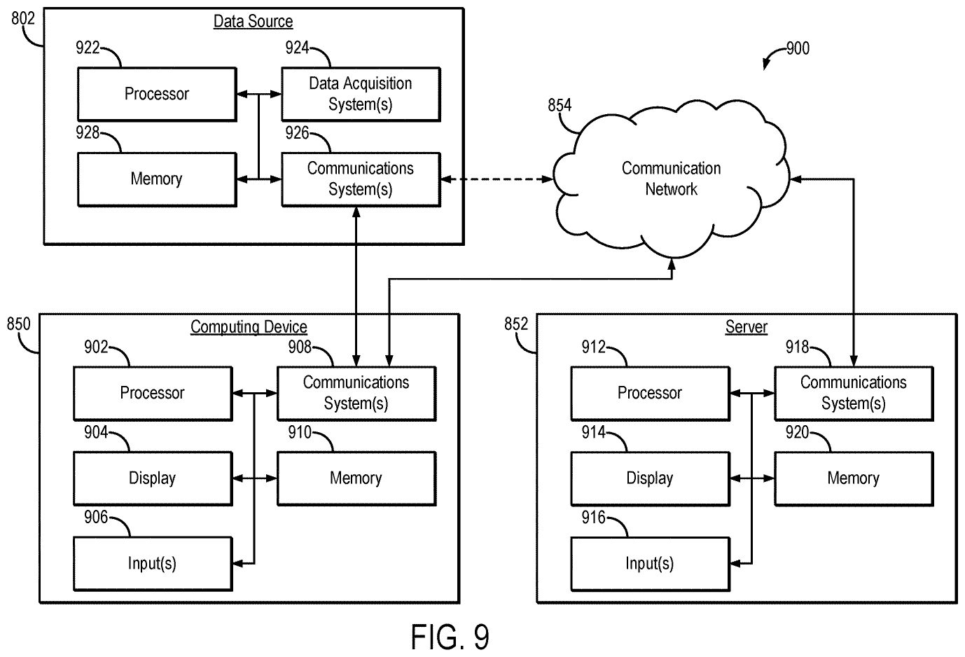

Referring now to FIG. 9, an example of hardware 900 that can be used to implement data source 802, computing device 850, and server 852 in accordance with some embodiments of the systems and methods described in the present disclosure is shown.

As shown in FIG. 9, in some embodiments, computing device 850 can include a processor 902, a display 904, one or more inputs 906, one or more communication systems 908, and/or memory 910. In some embodiments, processor 902 can be any suitable hardware processor or combination of processors, such as a central processing unit (“CPU”), a graphics processing unit (“GPU”), and so on. In some embodiments, display 904 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 906 can include any suitable input devices and/or sensors that can be used to receive user input, such as a keyboard, a mouse, a touchscreen, a microphone, and so on.

In some embodiments, communications systems 908 can include any suitable hardware, firmware, and/or software for communicating information over communication network 854 and/or any other suitable communication networks. For example, communications systems 908 can include one or more transceivers, one or more communication chips and/or chip sets, and so on. In a more particular example, communications systems 908 can include hardware, firmware, and/or software that can be used to establish a Wi-Fi connection, a Bluetooth connection, a cellular connection, an Ethernet connection, and so on.

In some embodiments, memory 910 can include any suitable storage device or devices that can be used to store instructions, values, data, or the like, that can be used, for example, by processor 902 to present content using display 904, to communicate with server 852 via communications system(s) 908, and so on. Memory 910 can include any suitable volatile memory, non-volatile memory, storage, or any suitable combination thereof. For example, memory 910 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 910 can have encoded thereon, or otherwise stored therein, a computer program for controlling operation of computing device 850. In such embodiments, processor 902 can execute at least a portion of the computer program to present content (e.g., images, user interfaces, graphics, tables), receive content from server 852, transmit information to server 852, and so on. For example, the processor 902 and the memory 910 can be configured to perform the methods described herein (e.g., the method of FIG. 1; the method of FIG. 2).

In some embodiments, server 852 can include a processor 912, a display 914, one or more inputs 916, one or more communications systems 918, and/or memory 920. In some embodiments, processor 912 can be any suitable hardware processor or combination of processors, such as a CPU, a GPU, and so on. In some embodiments, display 914 can include any suitable display devices, such as an LCD screen, LED display, OLED display, electrophoretic display, a computer monitor, a touchscreen, a television, and so on. In some embodiments, inputs 916 can include any suitable input devices and/or sensors that can be used to receive user input, such as a keyboard, a mouse, a touchscreen, a microphone, and so on.

In some embodiments, communications systems 918 can include any suitable hardware, firmware, and/or software for communicating information over communication network 854 and/or any other suitable communication networks. For example, communications systems 918 can include one or more transceivers, one or more communication chips and/or chip sets, and so on. In a more particular example, communications systems 918 can include hardware, firmware, and/or software that can be used to establish a Wi-Fi connection, a Bluetooth connection, a cellular connection, an Ethernet connection, and so on.

In some embodiments, memory 920 can include any suitable storage device or devices that can be used to store instructions, values, data, or the like, that can be used, for example, by processor 912 to present content using display 914, to communicate with one or more computing devices 850, and so on. Memory 920 can include any suitable volatile memory, non-volatile memory, storage, or any suitable combination thereof. For example, memory 920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 920 can have encoded thereon a server program for controlling operation of server 852. In such embodiments, processor 912 can execute at least a portion of the server program to transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone), and so on.

In some embodiments, the server 852 is configured to perform the methods described in the present disclosure. For example, the processor 912 and memory 920 can be configured to perform the methods described herein (e.g., the method of FIG. 1; the method of FIG. 2).

In some embodiments, data source 802 can include a processor 922, one or more data acquisition systems 924, one or more communications systems 926, and/or memory 928. In some embodiments, processor 922 can be any suitable hardware processor or combination of processors, such as a CPU, a GPU, and so on. In some embodiments, the one or more data acquisition systems 924 are generally configured to acquire patient health data, images, or both. Additionally or alternatively, in some embodiments, the one or more data acquisition systems 924 can include any suitable hardware, firmware, and/or software for coupling to and/or controlling operations systems for acquiring or collecting patient health data. In some embodiments, one or more portions of the data acquisition system(s) 924 can be removable and/or replaceable.

Note that, although not shown, data source 802 can include any suitable inputs and/or outputs. For example, data source 802 can include input devices and/or sensors that can be used to receive user input, such as a keyboard, a mouse, a touchscreen, a microphone, a trackpad, a trackball, and so on. As another example, data source 802 can include any suitable display devices, such as an LCD screen, an LED display, an OLED display, an electrophoretic display, a computer monitor, a touchscreen, a television, etc., one or more speakers, and so on.

In some embodiments, communications systems 926 can include any suitable hardware, firmware, and/or software for communicating information to computing device 850 (and, in some embodiments, over communication network 854 and/or any other suitable communication networks). For example, communications systems 926 can include one or more transceivers, one or more communication chips and/or chip sets, and so on. In a more particular example, communications systems 926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 928 can include any suitable storage device or devices that can be used to store instructions, values, data, or the like, that can be used, for example, by processor 922 to control the one or more data acquisition systems 924, and/or receive data from the one or more data acquisition systems 924; to generate images from data; present content (e.g., data, images, a user interface) using a display; communicate with one or more computing devices 850; and so on. Memory 928 can include any suitable volatile memory, non-volatile memory, storage, or any suitable combination thereof. For example, memory 928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 928 can have encoded thereon, or otherwise stored therein, a program for controlling operation of data source 802. In such embodiments, processor 922 can execute at least a portion of the program to generate images, transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating an electronic tumor marker from patient health data, the method comprising:
(a) accessing with a computer system, patient health data acquired from a patient;
(b) accessing with the computer system, a machine learning model that has been trained on training data in order to estimate an electronic tumor marker value from patient health data;
(c) inputting the patient health data to the machine learning model using the computer system, generating output data as electronic tumor marker data comprising electronic tumor marker values that are predictive of carbohydrate antigen 19-9 (CA19-9) serum values in the patient; and
(d) presenting the electronic tumor marker data to a user.

2. The method of claim 1, wherein the patient health data comprise electronic health record (EHR) data.

3. The method of claim 1, wherein the patient health data comprise clinical laboratory data.

4. The method of claim 1, wherein the patient health data comprise omics data.

5. The method of claim 1, wherein the patient health data comprise electronic health record (EHR) data and clinical laboratory data.

6. The method of claim 5, wherein the patient health data further comprise omics data.

7. The method of claim 1, wherein presenting the electronic tumor marker data to the user comprises generating a report based on the electronic tumor marker data and presenting the report to the user.

8. The method of claim 7, wherein the report indicates a predictive value of a medical condition in the patient based on the electronic tumor marker data.

9. The method of claim 8, wherein the medical condition is pancreatic cancer.

10. A non-transitory computer-readable storage media having stored thereon instructions that when executed by a processor cause the processor to perform a method comprising:
receiving patient health data with the processor, wherein the patient health data have been acquired from a patient;
receiving a machine learning model with the processor, wherein the machine learning model has been trained on training data to estimate an electronic tumor marker value from data consistent with the patient health data;
applying the patient health data to the machine learning model with the processor, thereby generating electronic tumor marker data as an output, wherein the electronic tumor marker data comprise electronic tumor marker values that are predictive of carbohydrate antigen 19-9 (CA19-9) serum values in the patient; and
outputting, by the processor, the electronic tumor marker data.

11. The non-transitory computer-readable storage media of claim 10, wherein outputting the electronic tumor marker data comprises generating a report that include the electronic tumor marker values in the electronic tumor marker data.

12. The non-transitory computer-readable storage media of claim 11, wherein the report further includes the patient health data.

13. The non-transitory computer-readable storage media of claim 11, wherein the report indicates a predictive value of a medical condition in the patient based on the electronic tumor marker data.

14. The non-transitory computer-readable storage media of claim 13, wherein the medical condition is pancreatic cancer.

15. The non-transitory computer-readable storage media of claim 10, wherein the patient health data comprise electronic health record (EHR) data.

16. The non-transitory computer-readable storage media of claim 10, wherein the patient health data comprise clinical laboratory data.

17. The non-transitory computer-readable storage media of claim 10, wherein the patient health data comprise omics data.

18. The non-transitory computer-readable storage media of claim 10, wherein the patient health data comprise electronic health record (EHR) data and clinical laboratory data.

19. The non-transitory computer-readable storage media of claim 18, wherein the patient health data further comprise omics data.
